(19) United States
(12) Patent Application Publication
(10) Pub. No.: US 2013/0142942 A1
(43) Pub. Date: Jun. 6, 2013

(54) Methods of Making a Reference Electrode for an Electrochemical Sensor

(71) Applicant: ABBOTT DIABETES CARE INC., Alameda, CA (US)

(72) Inventors: Yi Wang, San Ramon, CA (US); David A. Chang-Yen, Lake Villa, IL (US)

(73) Assignee: ABBOTT DIABETES CARE INC., Alameda, CA (US)

(21) Appl. No.: 13/678,110

(22) Filed: Nov. 15, 2012

Related U.S. Application Data
(60) Provisional application No. 61/561,135, filed on Nov. 17, 2011.

(51) Int. Cl. A61B 5/00

(52) U.S. Cl. CPC A61B 5/00
USPC 427/8; 427/593; 427/535

(57) ABSTRACT

Aspects of the present disclosure include methods for making an electrode for an electrochemical sensor. In practicing methods according to certain embodiments, a conductive layer is deposited on a substrate by high voltage electron beam thermal evaporation followed by depositing a reactive layer on a surface of the conductive layer by low-voltage resistive thermal evaporation using a sequential step, single production chamber. Also provided are methods for a producing a multi-layered reference electrode having silver or ITO and silver chloride thereon in the absence of a separate curing stage. Systems for practicing the subject methods are also described.

### CROSS-REFERENCE TO RELATED APPLICATIONS

Pursuant to 35 U.S.C. §119(e), this application claims priority to U.S. Provisional Patent Application No. 61/561,135 filed on Nov. 17, 2011, the disclosure of which is herein incorporated by reference in its entirety.

### INTRODUCTION

Management of diabetes requires knowledge of the glycemia of patients. In general, health care professionals and diabetic patients base their decisions of injection and dosage of insulin or ingestion of food on blood glycemia, meaning the glucose concentration in blood. In hospitals or clinics, venous blood is withdrawn and sent to a laboratory for analysis or is analyzed at the bedside or in the office of the health care professional. In some cases, the skin is lanced by the diabetic patient to obtain a droplet of blood which is used for a glucose assay such as with a glucose test strip system. Systems for frequently or continuously monitoring glycemia in the subcutaneous ISF, such as continuous glucose monitoring (CGM) devices, are also available.

Electrochemical analyte sensors are commonly used to determine the presence and concentration of an analyte in biological fluids. Some sensors are used to determine the concentration of glucose in a subject. Various methods of producing electrodes for electrochemical analyte sensors are available. Since monitoring the presence and concentration of analytes (e.g., glucose monitoring in diabetic patients) remain important in health care, practical and efficient methods for manufacturing analyte sensors are of significant interest.

### SUMMARY

Aspects of the present disclosure include methods for making an electrode for an electrochemical sensor. In practicing methods according to certain embodiments, a conductive layer is deposited on a substrate by high voltage electron beam thermal evaporation followed by depositing a reactive layer on a surface of the conductive layer by low-voltage resistive thermal evaporation using a sequential step, single production chamber. Also provided are methods for a producing a multi-layered reference electrode having silver or indium tin oxide (ITO) and silver chloride thereon in the absence of a separate curing stage. Systems for practicing the subject methods are also described.

In some embodiments, methods include making an electrode for an electrochemical sensor by applying a first material on a substrate by high-voltage electron beam thermal evaporation to produce a conductive layer and applying a second material onto the conductive layer by low-voltage resistive thermal evaporation to produce a reactive layer such that the conductive layer and the reactive layer are applied in the single chamber. In these embodiments, the first material may be a conductive compound selected from the group consisting of silver, indium tin oxide, gold, platinum, copper, nickel, rhodium, ruthenium, ruthenium dioxide, cobalt, zinc, titanium, palladium, carbon and platinum-carbon. In particular, the conductive layer may include silver or indium tin oxide Likewise, the second material may be a metal chloride, such as silver chloride. In certain embodiments, the conductive and the reactive layers are produced at room temperature. In other embodiments, the conductive and the reactive layers are produced in the absence of a separate curing step after depositing the conductive material or reactive layer material. In yet other embodiments, the conductive and reactive layers are deposited at reduced pressure, such as where the reaction chamber is maintained at a pressure of 5×10⁻⁶ torr or less, such as at a pressure of 1×10⁻⁶ torr or less. In embodiments of the present disclosure, the purity of the applied conductive and reactive layers may be 99% or greater, such as 99.9% or greater.

The conductive and reactive layer may be applied at any convenient rate, such as 2 Å per second to 10 Å per second, including at a rate of 2 Å per second to 5 Å per second. The reactive layer may be applied to the surface of the conductive layer after the conductive layer has reached a predetermined thickness, such as 10 nm or greater, including 100 nm or greater. Each layer may be applied by moving the substrate, such as by rotating the substrate or moving the substrate laterally in a back and forth motion while applying the first and second material.

In some embodiments, the substrate may be treated before applying the conductive layer. For example the substrate may be treated by a corona discharge process before applying the conductive layer. In other embodiments, an adhesion layer, such as a chromium adhesion layer is applied to the substrate before applying the conductive layer. The adhesion layer may be applied by any convenient protocol, such as for example high-voltage electron beam thermal evaporation.

In other embodiments, methods of the present disclosure include making an electrode for an electrochemical sensor by applying a first material on a substrate by high-voltage electron beam thermal evaporation to produce a conductive layer and applying a second material onto the conductive layer by low-voltage resistive thermal evaporation to produce a reactive layer, such that the second material is applied onto the conductive layer in the absence of a curing stage following application of the first material and such that the first and second material are applied in an single production chamber maintained at room temperature.

In other embodiments, methods of the present disclosure include making an electrode for an electrochemical sensor by applying a first material on a substrate by high-voltage electron beam thermal evaporation to produce a conductive layer and simultaneously applying the first material by high-voltage electron beam thermal evaporation and a second material by low-voltage resistive thermal evaporation onto the conductive layer to produce a multicomponent reactive layer.

### BRIEF DESCRIPTION OF THE DRAWINGS

A detailed description of various embodiments of the present disclosure is provided herein with reference to the accompanying drawings, which are briefly described below. The drawings are illustrative and are not necessarily drawn to scale. The drawings illustrate various embodiments of the present disclosure and may illustrate one or more embodiment(s) or example(s) of the present disclosure in whole or in part. A reference numeral, letter, and/or symbol that is used in one drawing to refer to a particular element may be used in another drawing to refer to a like element.

FIG. 1 shows a schematic of system for practicing methods according to certain embodiments of the present disclosure.

FIG. 2 illustrates a flowchart for producing an electrode having a conductive layer and a reactive layer according to one embodiment.

FIG. 3 illustrates a flowchart for producing an electrode having a conductive layer and a multicomponent reactive layer according to one embodiment.

### DETAILED DESCRIPTION

Aspects of the present disclosure include methods for making an electrode for an electrochemical sensor. In practicing methods according to certain embodiments, a conductive layer is deposited on a substrate by high voltage electron beam thermal evaporation followed by depositing a reactive layer on a surface of the conductive layer by low-voltage resistive thermal evaporation using a sequential step, single production chamber. Also provided are methods for a producing a multi-layered reference electrode having silver or ITO and silver chloride thereon in the absence of a separate curing stage. Systems for practicing the subject methods are also described.

Before the embodiments of the present disclosure are described, it is to be understood that this invention is not limited to particular embodiments described, as such may, of course, vary. It is also to be understood that the terminology used herein is for the purpose of describing particular embodiments only, and is not intended to be limiting, since the scope of the embodiments of the invention will be embodied by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

In the description of the invention herein, it will be understood that a word appearing in the singular encompasses its plural counterpart, and a word appearing in the plural encompasses its singular counterpart, unless implicitly or explicitly understood or stated otherwise. Merely by way of example, reference to “an” or “the” “analyte” encompasses a single analyte, as well as a combination and/or mixture of two or more different analytes, reference to “a” or “the” “concentration value” encompasses a single concentration value, as well as two or more concentration values, and the like, unless implicitly or explicitly understood or stated otherwise. Further, it will be understood that for any given component described herein, any of the possible candidates or alternatives listed for that component, may generally be used individually or in combination with one another, unless implicitly or explicitly understood or stated otherwise. Additionally, it will be understood that any list of such candidates or alternatives is merely illustrative, not limiting, unless implicitly or explicitly understood or stated otherwise.

Various terms are described below to facilitate an understanding of the invention. It will be understood that a corresponding description of these various terms applies to corresponding linguistic or grammatical variations or forms of these various terms. It will also be understood that the invention is not limited to the terminology used herein, or the descriptions thereof, for the description of particular embodiments. Merely by way of example, the invention is not limited to particular analytes, bodily or tissue fluids, blood or capillary blood, or sensor constructs or usages, unless implicitly or explicitly understood or stated otherwise, as such may vary.

The publications discussed herein are provided solely for their disclosure prior to the filing date of the application. Nothing herein is to be construed as an admission that the embodiments of the invention are not entitled to antedate such publication by virtue of prior invention. Further, the dates of publication provided may be different from the actual publication dates which may need to be independently confirmed.

In further describing the present disclosure, methods for producing an electrode for an electrochemical sensor are described first in greater detail. Next, devices and systems practicing methods of the present disclosure are also described.

### Methods for Producing and Electrode for an Electrochemical Sensor

As summarized above, aspects of the disclosure include methods for producing an electrode for an electrochemical sensor where a conductive layer is applied to the surface of a substrate and a reactive layer is applied on top of the conductive layer using a sequential step, single deposition chamber operating. By “sequential step” is meant that the reactive layer is applied directly on top of the conductive layer without deposition of any other material between the reactive layer and the conductive layer. By applying the reactive layer and the conductive layer in a sequential step process in a single deposition chamber according to methods of the present disclosure, large quantities of reactive layer material (e.g., silver chloride, AgCl) may be applied to the electrode surface. As such, large quantities of reactive materials may be accessible during sensor operation when the reference electrode is employed in an electrochemical sensor. In certain embodiments, a curing stage is not required in order to produce a functional electrode with deposited layers that exhibit substantial purity, smoothness and uniformity.

In some embodiments, methods of the present disclosure include applying a conductive layer to a substrate by electron beam thermal evaporation. As used herein, the term “applying” refers to placing one or more materials onto a surface, such as for example onto the surface of a substrate. As such, applying may include positioning on top, depositing or otherwise producing a material (e.g., conductive or nonconductive) on a surface. In certain embodiments, applying includes depositing a layer of material onto a surface. For example, methods may include depositing a thin layer of conductive material onto a surface, such as layer having a thickness of 1 nm or more, such as 2 nm or more, such as 5 nm or more, such as 10 nm or more, such as 25 nm or more, such as 50 nm or more and including 100 nm or more. In embodiments, material may be applied over the entire surface or a part of the surface, as desired. In some embodiments, applying material to a surface includes depositing material onto less than the entire surface. For instance, applying material to a surface may include depositing material onto 50% or less of the entire surface, such as 40% or less, such as 25% or less, such as 10% or less, such as 5% or less and including 1% or less of the entire surface. In certain instances, applying material to a surface includes depositing material to specific locations on the surface. For example, depositing material to specific locations may include depositing material onto the surface in the form of spots (or any other geometric shape) or strips (e.g., straight or non-straight having regular and irregular patterns).

The term “electron beam thermal evaporation” is used in its conventional sense to refer to the process for the deposition of an evaporant on a substrate by impinging a conductive material source with an electron beam to generate an evaporant and coating the substrate with the evaporant. As described in greater detail below, an electron beam produced by a charged filament is directed under high vacuum on one or more sources of conductive material to produce a conductive material in gaseous form (i.e., evaporant). The evaporant is then coated onto the surface of the substrate which forms a film of the conductive material on the substrate.

In embodiments of the present disclosure, the conductive layer is applied to a substrate by electron beam thermal evaporation. The source of the electron beam may be any convenient electron beam source, such as for example an electron beam gun (e.g., thermionic, photocathode, cold emission plasma source, etc.) or an electron beam emitter. Operating parameters of the electron beam source may vary depending on the conductive material employed and the deposited conductive layer desired. As such, the electron beam source may vary in applied voltage, operating watts, electron beam power, electron beam focus, electron beam pattern, scanning frequency and incident angle of the electron beam.

The composition of the conductive layer deposited to the surface of the substrate may vary depending on the conductive properties desired. The conductive layer may include but is not limited to carbon (e.g., graphite), conductive polymers, metals, alloys (e.g., gold, silver, titanium, platinum or any alloy thereof), or a metallic oxide composition (e.g., indium tin oxide (ITO) ruthenium dioxide or titanium dioxide). For example, the composition of the conductive layer may include but are not limited to aluminum, carbon (e.g., graphite), cobalt, copper, gallium, gold, indium, iridium, iron, lead, magnesium, mercury (as an amalgam), nickel, niobium, osmium, palladium, platinum, rhenium, rhodium, selenium, silicon (e.g., doped polycrystalline silicon), silver, tantalum, tin, titanium, tungsten, uranium, vanadium, zinc, zirconium, mixtures thereof, and alloys, oxides, or metallic compounds of these elements, for example indium tin oxide (ITO). In certain instances, the composition of the conductive layer is silver (Ag).

The conductive layer may include one or more of the aforementioned materials. For example, the conductive layer may include two or more components, such as three or more components, such as four or more components, including five or more components. In certain embodiments, the conductive layer includes only a single component. In these embodiments, the composition of the conductive layer contains a substantially pure composition of one component, as described in greater detail below. By “substantially pure” is meant that the composition of the conductive layer contains 99.5% or greater of a single material, such as 99.9% or greater, such as 99.99% or greater, such as 99.998% or greater of a single material. As such, the conductive layer includes 0.5% or less of any impurity, such as 0.1% or less, such as 0.05% or less, such as 0.01% or less, such as 0.005% or less, including 0.002% or less of any impurity. By “impurity” is meant any component of the conductive layer composition which is different from the desired conductive layer material and in some instances may be undesirable or is detrimental to the conductive layer composition. For example, impurities may interfere (i.e., diminish) or inhibit a particular desirable property of the conductive layer, such as for example conductivity. In other embodiments, impurities may be detrimental to the deposition consistency of the conductive layer, such as for example resulting in the conductive layer being unsuitable in thickness or smoothness. In certain embodiments, impurities may affect the surface of the applied conductive layer so as to make the conductive layer less suitable or altogether unsuitable for applying a reactive layer. Impurities may include, but are not limited to residual moisture, undesired metal constituents (including any aforementioned metals or alloys), conductive polymers, or trace materials which may mix with the evaporant during electron beam deposition. In certain instances, impurities may include oxidation of conductive layer material.

In embodiments of the present disclosure, the conductive material is placed in a crucible and vaporized using electron beam thermal evaporation, which deposits a thin film of conductive layer material onto the surface of a substrate. The crucible employed may vary depending on the conductive layer material and the required heating to produce an evaporant. For example, the crucible used for electron beam thermal evaporation may include, but is not limited to a standard graphite crucible, glassy coated graphite crucible, an alumina crucible, a boron crucible, a nitride crucible, a molybdenum crucible, a tantalum crucible or a tungsten crucible, among others. The dimensions of the crucible may vary depending on the size of the conductive layer and the electron beam source. For example the crucible may have a volume that ranges from 4 cubic centimeters (cc) to 200 cc, such as 5 to 175 cc, such as 10 cc to 150 cc, such as 25 cc to 100 cc, including 50 cc to 75 cc.

In some embodiments, the electron beam source for depositing the conductive layer is a high-voltage electron beam source. By “high-voltage” is meant that the electron beam source deposits the conductive layer while operating at a voltage which is 1000 V or greater, such as 2500 V or greater, such as 5000 V or greater, such as 7500 V or greater, such as 8400 V or greater, such as 10,000 V or greater, such as 15,000 V or greater, including 20,000 V or greater. In these instances, the electron beam source operates using an electron beam emitter source that is 1 kW or greater, such as 2 kW or greater, such as 3 kW or greater, such as 3.5 kW or greater, such as 5 kW or greater, such as 5.5 kW or greater, such as 6 kW or greater, such as 7.5 kW or greater, including 10 kW or greater. In certain embodiments, the electron beam emitter source uses a 6 kW closed-loop feedback-controlled emission filament operating at 8400V.

Depending on the deposition rate and conductive material applied, the beam pattern and scanning frequency of the electron beam source may vary. In some instances, the electron beam source employs a beam pattern having a diameter ranging from 0.00001 to 10 mm or greater, such as 0.0001 to 5 mm or greater, such as 0.001 to 1 mm or greater, such as 0.01 to 1 mm, including 0.1 to 1 mm or greater. For example, the electron beam pattern may have a diameter of 0.0001 mm or greater, such as 0.001 mm or greater, such as 0.01 mm or greater, including 0.1 mm or greater. The scanning pattern of the electron beam may also vary depending on the desired deposition, ranging from 25 to 1000 hertz, such as 50 to 900 hertz, such as 75 to 750 hertz, such as 100 to 500 hertz, including 150 to 450 hertz.

The beam energy required for deposition of the conductive layer varies depending on the conductive material and on the rate of vaporization desired. For example, the beam energy may be 0.01 MeV or greater, such as 0.1 MeV or greater, such as 1 MeV or greater, such as 5 MeV or greater, such as 10 MeV or greater, such as 100 MeV or greater, such as 250 MeV or greater, including 500 MeV or greater. In certain embodiments, the conductive material is silver. In these instances, the beam energy will range from 0.01 to 100 MeV, such as 0.1 to 90 MeV, such as 1 to 75 MeV, such as 10 to 50 MeV, including 10 to 25 MeV. In certain instances, the electron beam source is operated below the maximum vaporization rate to prevent melting instability of the conductive material which can result in ineffective vaporization or inconsistent conductive layer deposition. As such, the beam energy may be monitored and varied throughout the deposition process in order to ensure a uniform and consistent application of the conductive layer, as described in greater detail below. The vaporization rate of the conductive material may also vary, depending on the beam focus applied to the conductive material. The term “beam focus” is used in its conventional sense to refer to the beam peak characterized by the Gaussian radius (i.e., the radial distance from the centerline of the beam to the point where the energy density of the beam drops inversely of the peak value at the centerline). Depending on the conductive material, the beam focus may range from 2 to 200 mm, such as 5 to 150 mm, such as 10 to 100 mm, such as 15 to 90 mm, including 25 to 75 mm. As such, the specific beam focus may be controlled by varying the electrical current to the focusing coils of the beam source and may be monitored and varied as desired.

In embodiments of the disclosure, the conductive layer is deposited under reduced pressure. By “reduced pressure” is meant that the substrate is positioned in a sealed housing which has a pressure below atmospheric pressure. For example, the conductive layer may be applied at a pressure of 10⁻² torr or lower, such as 10⁻³, such as 10⁻⁴ torr or lower, such as 10⁻⁵ torr or lower, including as 10⁻⁶ torr or lower. In certain instances, the conductive layer is deposited under a high vacuum. By “high vacuum” is meant that the deposition chamber is evacuated to very low pressures, such as 10⁻⁷ torr or lower, such as 10⁻⁸ torr or lower and including 10⁻¹⁰ torr or lower. By applying the conductive layer under high vacuum, few impurities and unwanted particles become entrained in the gaseous stream during deposition resulting in an extremely high purity conductive layer. In some instances, the pressure of the sample chamber may be adjusted while the conductive layer is deposited. In other words, the pressure may be increased or decreased at any time during the deposition of the conductive layer. For example, in some instances the pressure of the sample chamber may be raised by 0.0000001 torr or more, such as by 0.000001 torr or more, such as by 0.0001 torr or more, such as by 0.001 torr or more, such as by 0.01 torr or more, including 0.1 torr or more. In other instances the pressure of the sample chamber is reduced by 0.0000001 torr or more, such as by 0.000001 torr or more, such as by 0.0001 torr or more, such as by 0.001 torr or more, such as by 0.01 torr or more, including 0.1 torr or more. The pressure of the sample chamber may be adjusted by any convenient method, including but not limited to mechanical roughing pumps, turbomolecular pumps, diffusion pump, among others.

In certain embodiments, the conductive layer is deposited onto the substrate by entraining the evaporant from the high-voltage electron beam thermal evaporation in a carrier gaseous stream. After impinging the conductive layer material with an electron beam producing an evaporant, a gas stream carries the evaporant to the substrate for deposition. Suitable carrier gases include, but are not limited to inert gases such as He, Argon, Ne and N₂, as well as other carrier gases such as O₂, hydrocarbons, silanes, methane and acetylene. In embodiments of the present disclosure, carrier gas streams contain substantially pure gas so as to minimize any contamination of the conductive layer during deposition. Accordingly, the carrier gas stream contains 0.1% or less by weight of any impurity, such as 0.01% or less, such as 0.001% or less, including 0.0001% or less by weight of any impurity.

In some embodiments, deposition of the conductive layer and the reactive layer (as described in detail below) is conducted at room temperature. The term “room temperature” is used in its conventional sense to refer to temperature of the ambient atmosphere, typically ranging between 20 to 25° C. (298K), such as 22° C. Since the present methods are capable, in certain instances, of producing functional conductive and reactive layers without requiring any heat curing steps (such as those required after screen printing or sputtering), each of the depositions of the conductive and reactive layers may be performed at room temperature. In other words, methods of the present disclosure include depositing one or more conductive layers and one or more reactive layers without heat curing either layer.

If desired however, the temperature of the deposition chamber during application of the conductive layer may be adjusted while the conductive layer is deposited. In other words, the temperature may be increased or decreased during deposition of the conductive layer. For example, in some instances the temperature may be raised by 10 K or more, such as by 25 K or more, such as by 50 K or more, such as by 100 K or more, such as by 500 K or more, including 1000 K or more. In other instances the temperature is reduced by by 10 K or more, such as by 25 K or more, such as by 50 K or more, such as by 100 K or more, such as by 500 K or more, including 1000 K or more.

The thickness of the conductive layer applied will depend on the conductive material, the rate of deposition, the number of layers applied and the duration of deposition. In some embodiments, the rate of deposition may range, such as from 0.01 to 500 Å/s, such as 0.1 to 250 Å/s, such as 1 to 100 Å/s, such as 10 to 90 Å/s, such as 15 to 75 Å/s, such as 20 to 60 Å/s, including 25 to 50 Å/s. The conductive layer may be applied for 0.5 seconds or longer, such as 1 second or longer, such as 2 seconds or longer, such as 5 seconds or longer, such as 10 seconds or longer, such as 30 seconds or longer, including 60 seconds or longer. One or more layers of conductive material may be applied to the substrate surface. For example, two or more layers of conductive material may be applied to the substrate surface, such as three or more layers, such as four or more layers, including 5 or more layers of conductive material may be applied to the substrate surface. As described in greater detail below, additional layers of conductive material may be added to the conductive layer if necessary, such as for example to improve smoothness and uniformity of the conductive layer. For example, if after evaluating the deposited conductive layer (by methods as described below), it is determined that the conductive layer is less than optimal or is unsuitable, additional conductive layers may be applied to all or part of the deposited conductive layer. As such, the thickness of the final deposited conductive layer may be 0.1 nm or more, such as 0.5 nm or more, such as 1.0 nm or more, such as 1.5 nm or more, such as 2.0 nm or more, such as 5 nm or more, such as 10 nm or more, including 100 nm or more. The amount of deposited conductive layer material will vary depending on the size of the applied area on the substrate as well as the number of layers deposited. In certain instances, the amount of conductive layer material applied is 100 ng or more, such as 250 ng or more, such as 500 ng or more, such as 1000 ng or more, including 2500 ng or more.

In some embodiments, the substrate is moved while applying the conductive layer. By “moved” is meant that movement is applied to the substrate in a regular pattern during application of the conductive layer. For example, the substrate may be rotated while the conductive layer is applied. In other instances, lateral movement may be applied to the substrate during application of the conductive layer.

In certain instances, the substrate is rotated during application of the conductive layer. For example, the substrate may be rotated continuously during application. By “rotated continuously” is meant that the substrate rotates either clockwise or counterclockwise without a change in direction at any time during application of the conductive layer. For example, the substrate may be rotated continuously in a clockwise direction as the conductive layer is deposited. In other instances, the substrate is rotated continuously in a counterclockwise direction as the conductive layer is deposited. The rotation rate of the substrate while the conductive layer is deposited may vary, ranging from 1×10⁻³ to 1×10⁵ rps (revolutions per second), such as from 5×10⁻² to 1×10⁵ rps, such as from 1×10⁻² to 5×10⁴ rps, such as from 5×10⁻¹ to 1×10³ rps, such as 1 to 5×10² rps, including 5 to 10 rps. Any convenient protocol can be used to rotate the substrate while depositing the conductive layer, such as for example by an electric motor, an electromagnetic rotation device, among others.

In other instances, the substrate may be rotated in a reciprocating motion. By “reciprocating motion” is meant the substrate is rotated in an alternating fashion such that the substrate rotates in one direction (e.g., clockwise) for a first predetermined period of time and changes direction to rotate in the opposing direction (e.g., counterclockwise) for second predetermined period of time. For example, the substrate may be rotated in a “back-and-forth” motion, alternating between clockwise and counterclockwise motion. Each direction (e.g., clockwise or counterclockwise) can be performed for any amount of time as desired. For example, the substrate may be rotated in either direction for 10⁻³ seconds or more, such as 10⁻² seconds or more, such as 10⁻¹ seconds or more, such as 1 second or more, such as 2 seconds or more, such as 5 seconds or more, such as 10 seconds or more, such as 100 seconds or more, including 500 seconds or more. The rate of rotation in either direction may be the same or different, as desired. The rate of rotation in either direction may be constant (i.e., stays the same throughout application of the conductive layer) or may be variable (i.e., changes at any time during application of the conductive layer). Furthermore, the reciprocating motion may be repeated as desired, such as 2 times or more, such as 5 times or more, such as 10 times or more, such as 50 times or more, such as 100 times or more, such as 1000 times or more, such as 10,000 times or more, including 100,000 times or more.

In other embodiments, lateral movement is applied to the substrate while the conductive layer is deposited. By “lateral movement” is meant the substrate is moved in a back and forth motion such that a particular location on the substrate may move a predetermined distance, come to a stop and return to its original location. Lateral movement can be made in any direction, such as vertically, horizontally, or any combination thereof (i.e., diagonally with respect to the midline of the substrate). The amplitude or total displacement of the substrate may vary. By “amplitude of displacement” or “total displacement” is meant the sum total of distance traversed by a particular location (e.g., midline) on the substrate during movement. For example, lateral movement applied to a substrate which has a total displacement of 2 mm is meant the location traverses a total of 2 mm during the lateral movement. For example, the location may move 2 mm from the initial location and come to a stop resulting in a 2 mm total displacement or the location may move 1 mm from the initial location and move a second 1 mm to return to its initial location. In embodiments of the present disclosure, lateral movement of the substrate when applying the conductive layer may vary, the amplitude of displacement ranging from about 10 to 50 mm, such as from about 15 to 45 mm, such as from about 15 to 40 mm, such as from about 15 to 35, such as from about 20 to 30 mm, including from about 22 to 25 mm. The rate of lateral movement may vary. For example, the back and forth movement of the substrate may range from about 1 to 25 times per second, such as 5 to 25 times per second, such as 10 to 20 times per second, including 15 times per second.

The temperature of the substrate during application of the conductive layer may vary, ranging such as from −150° C. to 1500° C., such as from −100° C. to 1250° C., such as from −50° C. to 1000° C., such as from 0° C. to 750° C., such as from 100° C. to 500° C., including 250° C. to 400° C. In certain embodiments, the temperature of the substrate is equivalent to the internal temperature of the application chamber. In these instances the temperature of the substrate is not changed and remains at room temperature throughout the entire deposition process. If desired, the temperature of the substrate may be modified at any time during the deposition of the conductive layer. In other words, the temperature of the substrate may be increased or decreased at any time while the conductive layer is deposited to the substrate. As such, the temperature of the substrate may be increased or decreased by 0.01° C. or more, such as 0.05° C. or more, such as 0.1° C. or more, such as 0.5° C. or more, such as 1° C. or more, such as 5° C. or more, such as 10° C. or more, such as 25° C. or more, such as 50° C. or more, such as 100° C. or more, including 250° C. or more. The temperature may also be maintained at a constant temperature. The temperature of the substrate may be modified by any convenient protocol, so long as it can cool or heat the substrate and may include by is not limited to thermal heat exchangers, electric heating coils, Peltier thermoelectric devices, coils employing refrigerants, coils employing cryogenic fluids, among other protocols.

In certain instances, the conductive layer evaporant may be conditioned prior to depositing the conductive layer onto the substrate. By “conditioned” is meant that the conductive material is degassed, heated, purified or otherwise prepared for deposition. In certain instances, a shutter is placed above the crucible and the evaporant is conditioned and monitored for any spitting characteristics prior to deposition. The extent and amount of conditioning depends on the type of conductive material and its purity. For example, employing substantially pure conductive materials (i.e., greater than 99.5% pure) may reduce the number and duration of required conditioning steps.

In some embodiments, prior to applying the conductive layer, the substrate surface is conditioned for applying the conductive layer. In certain instances, the substrate is treated with a corona discharge process. The term “corona discharge process” is used herein in its conventional sense to refer to a surface treatment process which prepares a surface to be more receptive to an applied coating (e.g., conductive layer). In some embodiments, the substrate is treated with a corona discharge surface treatment by applying a potential across electrodes sufficient to generate a discharge between the electrodes under a gas atmosphere. As such, the substrate can be treated without the use of any liquid solvent. Any convenient gas source may be employed sufficient to produce the corona discharge. For example, the gas may be air, O₂, water vapor, CO₂, oxygen-containing organic gases such as alcohols, ketones, ethers and any combination thereof. Gases for corona discharge treatment may also include nitrogen containing gases, such as N₂ and ammonia. In other instances, the corona discharge process may be carried out using halogenated gases, such as for example, F₂, Cl₂, Br₂, I₂, HF, HCl, HBr and HI, CF₄, CHClF₂, CClF₃, CCl₂F₂, C₂F₆, CBrF₃, CHCl₃, CH₂Cl₂, CH₃CCl₃, CCl₄, or any combination thereof. Suitable corona discharge surface treatment protocols may also include, but are not limited, surface corona discharge processing described in U.S. Pat. Nos. 4,358,681; 4,879,100; 5,194,291; 5,236,536 and 5,466,424, the disclosures of which are herein incorporated by reference.

Surface treatment by corona discharge process conditions or prepares the substrate surface to be more receptive to the applied conductive layer. By “more receptive” is meant that corona discharge process improves the deposition (e.g. adhesion, surface smoothness, etc.) of the conductive layer to the surface of the substrate. For example, in certain instances, the corona discharge process increases the hydrophilicity of the substrate surface. For example, the corona discharge process increases the hydrophilicity of the substrate surface by 2 times or more, such as 3 times or more, such as 5 times or more, including 10 times or more. In other instances, the corona discharge process increases the hydrophobicity of the substrate surface. For example, the corona discharge process increases the hydrophobicity of the substrate surface by 2 times or more, such as 3 times or more, such as 5 times or more, including 10 times or more. The hydrophilicity or hydrophobicity of the substrate surface may be measured by any convenient protocol, such as for example measuring surface contact angles, flow microcalorimetry and the like. In other instances, the corona discharge process increases the adhesion of the substrate surface to the applied conductive layer. For example, the corona discharge process increases the adhesion of the substrate surface by 2 times or more, such as 3 times or more, such as 5 times or more, including 10 times or more.

Surface treatment by corona discharge process may vary depending on the substrate. Substrates as described herein may include, but are not limited to flexible or rigid plastic, polymeric or thermoplastic materials, such as for example polycarbonates, polyesters (e.g., Mylar™ and polyethylene terephthalate (PET)), polyvinyl chloride (PVC), polyurethanes, polyethers, polyamides, polyimides, or copolymers of these thermoplastics, such as PETG (glycol-modified polyethylene terephthalate). Other examples of substrates include substrates as described in detail in U.S. Pat. Nos. 6,175,752 and 6,565,509, the disclosures of which are herein incorporated by reference.

Corona discharge process may be conducted at pressures which range from 0.001 to 4000 torr, such as 0.01 to 4000 torr, such as 0.1 to 4000 torr, such as 1 to 2500 torr, such as 5 to 1000 torr, such as 10 to 750 torr, including from 25 to 750 torr. In certain instances, corona discharge treatment is conducted at pressures equivalent or less than the pressure at which the conductive layer will be deposited to the substrate. As such, corona discharge treatment may be conducted at reduced pressures, such as a pressure of 10⁻² torr or lower, such as 10⁻³, such as 10⁻⁴ torr or lower, such as 10⁻⁵ torr or lower, including 10⁻⁶ torr or lower. In certain embodiments, the corona discharge process is conducted under high vacuum, such as at a deposition chamber pressure of 10⁻⁷ torr or lower, such as 10⁻⁸ torr or lower, including 10⁻¹⁰ torr or lower. Conversely, the corona discharge process may be conducted at pressures which are greater than the pressure for deposition of the conductive layer, such for example, at a pressure of 10⁻⁶ torr or greater, such as 10⁻⁵ torr or greater, such as 10⁻⁴ torr or greater, such as 10⁻³ torr or greater, including 10⁻² torr or greater. In certain embodiments, the pressure of the sample chamber may be adjusted during corona discharge treatment. In other words, the pressure may be increased or decreased at any time during corona discharge treatment. For example, the pressure of the sample chamber may be increased by 10⁻⁶ torr or more, such as 10⁻⁵ torr or more, such as 10⁻⁴ torr or more, such as 10⁻³ torr or more, including 10⁻² torr or more. In other instances the pressure of the sample chamber is decreased by 10⁻⁶ torr or more, such as 10⁻⁵ torr or more, such as 10⁻⁴ torr or more, such as 10⁻³ torr or more, including 10⁻² torr or more.

The surface of the substrate may be treated by corona discharge process one or more times as desired, such as 2 or more times, such as 3 or more times, including 5 or more times before application of the conductive layer. In certain embodiments, the substrate surface is evaluated during or following corona discharge process. By “evaluated” is meant that certain properties of the substrate surface may be determined for suitability for depositing the conductive layer. For example, the smoothness and uniformity of the substrate surface, hydrophilicity, hydrophobicity or metal adhesion properties may be assessed. Any convenient protocol may be employed to evaluate the substrate surface. Methods for evaluating the substrate surface may include, but are not limited to electron microscopy, atomic force microscopy, diffuse reflectance spectrophotometry, flow microcalorimetry, quartz crystal microbalance, contact angle analysis, adhesion studies, among others.

The substrate surface may be evaluated at any phase during the corona discharge process. For example, the substrate surface may be evaluated before and after corona discharge treatment. In certain instances, methods include monitoring the substrate surface throughout the entire procedure. For example, evaluating the substrate surface may include collecting real-time data (e.g., hydrophilicity, smoothness). In other embodiments, the substrate surface is evaluated at regular intervals, e.g., determining the hydrophilicity of the substrate surface every 1 minute, every 5 minutes, every 10 minutes, every 30 minutes, every 60 minutes, every 100 minutes, every 200 minutes, every 500 minutes or some other interval. Methods of the present disclosure also include assessing the substrate surface after corona discharge treatment and before depositing the conductive layer to the substrate surface. By “assessing” the substrate surface is meant that a human (either alone or with the assistance of a computer, if using a computer-automated process initially set up under human direction), evaluates the substrate surface and determines whether the substrate surface is suitable or unsuitable for applying the conductive layer. If after assessing that the substrate surface is suitable following corona discharge treatment for depositing the conductive layer, the conductive layer may be applied to the substrate surface without further adjustments. In other words, methods of these embodiments include a step of assessing the evaluated substrate surface to identify any desired adjustments to the substrate surface. The desired adjustments may vary in terms of goal, where in some instances the adjustments are adjustments that ultimately result in enhanced performance of some desirable parameter, e.g., energy required to deposit the conductive layer, smoothness of the deposited conductive layer or thickness of the deposited conductive layer. In some instances, where the substrate surface has been determined to be at least less than optimal for applying the conductive layer, the substrate surface may be further processed. Where desired, the substrate surface may be conditioned by corona discharge process one or more times. In some embodiments, it may be determined that no adjustment of the substrate surface is desired or necessary.

Processing the substrate surface after corona discharge treatment may include adjusting (e.g., increasing or decreasing) the hydrophilicity of the substrate. For instance, processing the substrate surface may include making the substrate more hydrophilic. As such, the hydrophilicity of the substrate surface may be increased, such as 1.5 times or greater, such as 2 times or greater, such as 3 times or greater, such as 5 times or greater, including 10 times or greater.

Processing the substrate surface after corona discharge treatment may also include adjusting (e.g., increasing or decreasing) the metal adhesion of the substrate. For instance, processing the substrate surface may include increasing the adhesion of the substrate surface to a deposited metal. As such, the metal adhesion of the substrate surface may be increased such as 1.5 times or greater, such as 2 times or greater, such as 3 times or greater, such as 5 times or greater, including 10 times or greater.

The hydrophilicity, hydrophobicity or metal adhesion of the substrate surface may be adjusted using any convenient protocol, such as for example, plasma treatment (e.g., microwave plasmas or low pressure plasmas), additional corona discharge treatment, electrowetting, by application of a hydrophilic thin film, among other treatment protocols. Plasma treatments for increasing the hydrophilicity of the substrate surface may include, but are not limited to oxygen plasmas, carbon dioxide plasmas, NO plasmas, NO₂ plasmas, or halogen containing gases plasmas and the like.

In some instances, the conductive layer may be applied to the substrate immediately after corona discharge surface treatment. In other instances, the conductive layer is deposited to the substrate after a predetermined period after corona discharge treatment. For example, the conductive layer may be applied to the substrate, 1 second or more after surface treatment, such as 2 seconds or more, such as 5 seconds or more, such as 10 seconds or more, such as 60 seconds or more, including 100 seconds or more after corona discharge treatment. In certain instances, the treated substrate may be stored for a period of time before applying the conductive layer to the substrate. In certain instances, the substrate may be stored for 1 to 1000 days or longer, such as 1 to 100 days or longer, including 1 to 10 days or longer. Any storage method may be employed so long as it is sufficient to store the treated substrate without changing any of the desired properties of the substrate. In certain instances, the substrate is stored in an evacuated chamber directly linked to the surface treatment chamber. In other instances, the treated substrate is stored in the conductive layer deposition chamber, such as remaining affixed to the substrate holder.

Conditions for storing the treated substrate may vary. In certain embodiments, the treated substrate is stored under reduced pressure, such as for example at a pressure of 10⁻² torr or lower, such as 10⁻³, such as 10⁻⁴ torr or lower, such as 10⁻⁵ torr or lower, such as 10⁻⁶ torr or lower, such as 10⁻⁷ torr or lower, including 10⁻⁸ torr or lower. In other instances, the treated substrate may be stored in an unreactive gas sample. The term “unreactive gas sample” is used in its conventional sense to refer to a gaseous atmosphere which does not result in any type of chemical interaction with the substrate. For example, the treated substrate may be stored under a N₂ or argon gaseous atmosphere. In certain embodiments, prior to applying the conductive layer, an adhesion layer is applied to the surface of the substrate. The adhesion layer may be applied with or without first treating the substrate surface by a corona discharge process, as described above. In embodiments where the substrate surface is first treated by a corona discharge process, an adhesion layer may be applied immediately after treatment of the substrate. In yet other embodiments, the adhesion layer is applied after the treated substrate has been stored for a predetermined period of time, as described above.

The adhesion layer applied to the substrate surface serves to improve the contact between the conductive layer and the substrate, preventing detachment of the conductive material from the substrate, thus improving electrode performance and prolonging electrode lifetime. In some instances, the adhesion layer is a metal adhesion layer, such as for example a chromium adhesion layer. The thickness of the adhesion layer may vary, depending on the size and desired properties of the electrode. For example, the thickness of the adhesive layer may be 0.01 Å or more, such as 0.05 Å or more, such as 0.1 Å or more, such as 0.1 Å or more, such as 0.5 Å or more, such as 1 Å or more, such as 1.5 Å or more, such as 2 Å or more, such as 5 Å or more, including 10 Å or more. The thickness of part or all of the adhesion layer maybe adjusted at any time before the conductive layer is applied onto the adhesive layer. For example, in some embodiments, methods include increasing the thickness of the entire adhesion layer. In other embodiments, less than that entire adhesive layer may be increased in thickness, such as 95% or less of the adhesive layer is increased in thickness, such as 75% or less, such as 50% or less, such as 25% or less, such as 10% or less, and including 5% or less of the adhesion layer is increased in overall thickness. As such, all or part of the adhesion layer be increased by 0.01 Å or more, such as by 0.05 Å or more, such as by 0.1 Å or more, such as by 0.1 Å or more, such as by 0.5 Å or more, such as by 1 Å or more, such as by 1.5 Å or more, such as by 2 Å or more, such as by 5 Å or more, including by 10 Å or more.

Methods of the present disclosure also include depositing a reactive layer on top of the conductive layer by low-voltage resistive thermal evaporation in a sequential step following deposition of the conductive layer. The term “low-voltage resistive thermal evaporation” is used herein to refer to a process for the deposition of reactive layer material on top of the conductive layer by heating a material source using a low-voltage resistive heating element to generate an evaporant and coating the conductive layer with the evaporant. As described in greater detail below, the reactive layer material is vaporized by indirectly applying heat to a crucible containing the reactive layer material. The evaporant is then coated onto the surface of the conductive layer which forms the reactive layer.

In some embodiments, the reactive layer is deposited on top of the conductive layer by low-voltage resistive thermal evaporation. By “low-voltage” is meant that the low-voltage resistive thermal evaporation source deposits the reactive layer while operating at a voltage which is 10 V or less, such as 9 V or less, such as 8 V or less, such as 7 V or less, such as 5 V or less, such as 3 V or less, such as 2 V or less, including 1 V or less. In these instances, the low-voltage resistive thermal evaporation source uses a closed-loop feedback-controlled resistive crucible heater that operates at 2 kW or less, such as 1.5 kW or less, such as 1 kW or less, such as 0.5 kW or less, such as 0.25 kW or less, including 0.1 kW or less. In certain embodiments, the low-voltage resistive thermal evaporation source uses a 2 kW closed-loop feedback-controlled resistive crucible heater operating at less than 10V.

The reactive layer is composed of a deposited reactive layer material. The reactive layer material may include but is not limited to a halogenated metal salt (e.g., I, Br, Cl, F) such as chlorine or bromine combined with aluminum, cobalt, copper, gallium, gold, indium, iridium, iron, lead, magnesium, mercury (as an amalgam), nickel, niobium, osmium, palladium, platinum, rhenium, rhodium, selenium, silver, tantalum, tin, titanium, tungsten, uranium, vanadium, zinc or zirconium. For example, the composition of the reactive layer is, in certain embodiments, silver chloride (AgCl). In other embodiments, the composition of the reactive layer is a mixture of silver and silver chloride.

In some embodiments, the reactive layer may include one or more of the aforementioned compounds. For example, the reactive layer may include two or more compounds, such as three or more compounds, such as four or more compounds, including five or more compounds. In certain instances, the reactive layer includes only a single compound. In these instances, the composition of the reactive layer contains a substantially pure composition of one compound, such as for example, substantially pure silver chloride. As described above, a substantially pure composition as described herein is meant that the composition contains 99.5% or greater of the desired reactive layer material, such as 99.9% or greater, such as 99.99% or greater, such as 99.998% or greater of the desired reactive layer material. For example, a pure composition may include 99.998% silver chloride (AgCl). As such, the reactive layer includes 0.5% or less of any impurity, such as 0.1% or less, such as 0.05% or less, such as 0.01% or less, such as 0.005% or less, including 0.002% or less of any impurity. An impurity in the reactive layer is meant any component of the reactive layer composition which differs from the reactive layer material that may be undesirable or detrimental to the reactive layer composition. For example, impurities may interfere (i.e., diminish) or inhibit a particular desirable property of the reactive layer, such as for example conductivity, thickness, uniformity of the reactive layer or smoothness of the reactive layer.

Aspects of the present disclosure include placing the reactive layer material into a crucible and vaporizing the reactive layer material by a closed-loop feedback-controlled resistive crucible heater, which deposits the reactive layer material onto the deposited conductive layer. The crucible employed may vary depending on the reactive layer material employed and the heat required to produce the evaporant. For example, the crucible may include, but is not limited to a quartz-crystal crucible, a standard graphite crucible, glassy coated graphite crucible, an alumina crucible, a boron crucible, a nitride crucible, a molybdenum crucible, a tantalum crucible or a tungsten crucible, among others. In certain embodiments, the crucible is a quartz-crystal crucible. The dimensions of the crucible may vary depending on the amount of reactive layer deposited. For example the crucible may have a volume that ranges from 4 cubic centimeters (cc) to 200 cc, such as 5 to 175 cc, such as 10 cc to 150 cc, such as 25 cc to 100 cc, including 50 cc to 75 cc.

The temperature of the feedback-controlled resistive crucible heater during deposition of the reactive layer may vary depending on the type of material deposited and the desired rate of deposition. In some instances, the resistive heater heats the crucible containing the reactive layer material to a temperature that is 500° C. or greater, such as 750° C. or greater, such as 1000° C. or greater, such as 1250° C. or greater, such as 1500° C. or greater, such as 2000° C. or greater, including 2500° C. or greater. The temperature may be adjusted at any time during deposition of the reactive layer. In other words, the temperature may be increased or decreased during the deposition process. For example, in some instances the temperature may be increased or reduced by 10° C. or more, such as by 25° C. or more, such as by 50° C. or more, such as 75° C. or more, such as by 100° C. or more, such as by 250° C. or more, including 500° C. or more. In certain instances, the low-voltage resistive crucible heater is operated below the maximum vaporization rate to prevent melting instability of the reactive layer material which can result in ineffective vaporization or inconsistent reactive layer deposition. As such, the resistive crucible heater may be monitored and varied throughout the deposition process in order to ensure a uniform and consistent application of the reactive layer, as described in greater detail below.

In some embodiments, the reactive layer may also be applied under reduced pressure, such as described above for the conductive layer. For example, the reactive layer may be applied at a pressure of 10⁻² torr or lower, such as 10⁻³, such as 10⁻⁴ torr or lower, such as 10⁻⁵ torr or lower, including as 10⁻⁶ torr or lower. In certain instances, the reactive layer is applied under a high vacuum. By “high vacuum” is meant that the deposition chamber is evacuated to very low pressures, such as 10⁻⁷ torr or lower, such as 10⁻⁸ torr or lower and including 10⁻¹⁰ torr or lower. The deposition chamber may have the same or different pressure during application of the reactive layer as during application of conductive layer.

By depositing the reactive layer under reduced pressure or under a high vacuum, few impurities and unwanted particles become entrained during deposition resulting in an extremely high purity reactive layer. In some instances, the pressure of the sample chamber may be adjusted while the reactive layer is deposited. For example, in some instances the pressure of the sample chamber may be raised or reduced by 0.0000001 torr or more, such as by 0.000001 torr or more, such as by 0.0001 torr or more, such as by 0.001 torr or more, such as by 0.01 torr or more, including 0.1 torr or more.

In certain embodiments, the reactive layer is deposited onto the substrate by entraining the evaporant from the low-voltage resistive heating thermal evaporation in a carrier gaseous stream. After resistive heating of the crucible carrying the reactive layer material producing an evaporant, a gas stream carries the evaporant to the surface of the conductive layer for deposition. Suitable carrier gases include, but are not limited to inert gases such as He, Argon, Ne and N₂, as well as other carrier gases such as O₂, hydrocarbons, silanes, methane and acetylene. In embodiments of the present disclosure, carrier gas streams contain substantially pure gas so as to minimize any contamination of the reactive layer during deposition. Accordingly, the carrier gas stream contains 0.1% or less by weight of any impurity, such as 0.01% or less, such as 0.001% or less, including 0.0001% or less by weight of any impurity.

The temperature of the deposition chamber during application of the reactive layer may be the same or different from the temperature when depositing the conductive layer. Aspects of the present disclosure may also include depositing the reactive layer at room temperature. Since the present methods are capable of producing functional conductive and reactive layers without requiring any heat curing steps (such as those required after screen printing or sputtering), each of the deposition reactive layer maybe performed at room temperature. In certain embodiments, the reactive layer is deposited at room temperature and no additional heat is applied to the reactive layer after deposition.

In certain embodiments, no heat or curing of any type is required to complete deposition of either the conductive layer or reactive layer. By “no heat or curing step” is meant methods of the present disclosure do not require that either the conductive or reactive layers be heated or otherwise treated after deposition in order to produce a functional conductive or reactive layer. As such, after depositing the conductive or reactive layer, no additional heating, chemical or physical treatment steps are required. In these embodiments, each layer is ready immediately for the next step of processing as soon as the material is deposited. For example, the reactive layer may be deposited onto the surface of the conductive layer immediately after deposition of the conductive layer. Likewise, immediately after deposition of the reactive layer by low-voltage thermal evaporation, the reactive layer is considered to be complete and the electrode is ready for use.

If necessary, the temperature may be adjusted during deposition of the reactive layer. In other words, the temperature may be increased or decreased during the deposition of the reactive layer. For example, in some instances the temperature may be raised or reduced by 10 K or more, such as by 25 K or more, such as by 50 K or more, such as by 100 K or more, such as by 500 K or more, including 1000 K or more. In certain instances, the temperature is maintained constant during deposition of the reactive layer.

The thickness of the deposited reactive layer will depend on the reactive layer material, the rate of deposition, the number of layers deposited and the duration of deposition. The rate of deposition of the reactive layer by low-voltage resistive evaporation may range from 0.01 to 500 Å/s, such as 0.1 to 250 Å/s, such as 1 to 100 Å/s, such as 10 to 90 Å/s, such as 15 to 75 Å/s, such as 20 to 60 Å/s, including 25 to 50 Å/s. The reactive layer may be applied for 0.5 seconds or longer, such as 1 second or longer, such as 2 seconds or longer, such as 5 seconds or longer, such as 10 seconds or longer, such as 30 seconds or longer, including 60 seconds or longer. One or more layers of the reactive layer material may be deposited on top of the conductive layer. For example, two or more layers of reactive layer material may be deposited on top of the conductive layer, such as three or more layers, such as four or more layers, including 5 or more layers of reactive layer material may be deposited on top of the conductive layer. As described in greater detail below, additional layers may be added to the reactive layer if necessary, such as for example to improve smoothness and uniformity. For example, if after evaluating the deposited reactive layer (by methods as described below), it is determined that the reactive layer is at least less than optimal or is unsuitable, addition reactive layers may be applied to all or part of the deposited reactive layer. As such, the thickness of the applied reactive layer may be 0.1 nm or more, such as 0.5 nm or more, such as 1.0 nm or more, such as 1.5 nm or more, such as 2.0 nm or more, such as 5nm or more, such as 10 nm or more, including 100 nm or more. The amount of deposited reactive layer material will vary depending on the size of the applied area as well as the number of layers deposited. In certain instances the amount of reactive layer material applied is 100 ng or more, such as 250 ng or more, such as 500 ng or more, such as 1000 ng or more, including 2500 ng or more.

In some embodiments, movement is applied to the substrate while the reactive layer is applied. In certain instances, the substrate is rotated during application of the reactive layer. For example, the substrate with applied conductive layer may be rotated continuously in a clockwise direction as the reactive layer is applied on top of the conductive layer. In other instances, the substrate with applied conductive layer is rotated continuously in a counterclockwise direction as the reactive layer is applied. The rotation rate may vary, ranging from 1×10⁻³ to 1×10⁵ rps (revolutions per second), such as from 5×10⁻² to 1×10⁵ rps, such as from 1×10⁻² to 5×10⁴ rps, such as from 5×10⁻¹ to 1×10³ rps, such as 1 to 5×10² rps, including 5 to 10 rps.

In other instances, the substrate may be rotated in a reciprocating motion. Each direction (e.g., clockwise or counterclockwise) can be performed for any amount of time as desired. For example, the substrate may be rotated in either direction for 10⁻³ seconds or more, such as 10⁻² seconds or more, such as 10⁻¹ seconds or more, such as 1 second or more, such as 2 seconds or more, such as 5 seconds or more, such as 10 seconds or more, such as 100 seconds or more, including 500 seconds or more. The rate of rotation in either direction may be the same or different, as desired. The rate of rotation in either direction may be constant or may be variable. Furthermore, the reciprocating motion may be repeated as desired, such as 2 times or more, such as 5 times or more, such as 10 times or more, such as 50 times or more, such as 100 times or more, such as 1000 times or more, such as 10,000 times or more, including 100,000 times or more.

In other embodiments, lateral movement is applied to the substrate while the reactive layer is applied. Lateral movement can be made in any direction, such as vertically, horizontally, or any combination thereof (i.e., diagonally with respect to the midline of the substrate). The amplitude or total displacement of the substrate may vary. In embodiments of the present disclosure, lateral movement of the substrate when applying the reactive layer may vary, the amplitude of displacement ranging from about 10 to 50 mm, such as from about 15 to 45 mm, such as from about 15 to 40 mm, such as from about 15 to 35, such as from about 20 to 30 mm, including from about 22 to 25 mm. The rate of lateral movement may vary. For example, the back and forth movement of the substrate may range from about 1 to 25 times per second, such as 5 to 25 times per second, such as 10 to 20 times per second, including 15 times per second.

The temperature of the substrate during application of the reactive layer may be the same or different as when applying the conductive layer. In certain instances, the substrate temperature remains constant throughout application of the conductive and reactive layers. In other instances, the temperature differs, such as by 10° C. or more, such as by 25° C. or more, such as by 50° C. or more, such as by 75° C. or more, including by 100° C. or more. In certain embodiments, the temperature of the substrate is equivalent to the internal temperature of the application chamber. In these instances, the temperature of the substrate is not changed and remains at room temperature throughout the entire deposition process. If desired, the temperature of the substrate may be modified at any time during the deposition of the reactive layer. In other words, the temperature of the substrate may be increased or decreased at any time while the reactive layer is deposited to the substrate. As such, the temperature of the substrate may be increased or decreased by 0.01° C. or more, such as 0.05° C. or more, such as 0.1° C. or more, such as 0.5° C. or more, such as 1° C. or more, such as 5° C. or more, such as 10° C. or more, such as 25° C. or more, such as 50° C. or more, including 100° C. or more.

Aspects of the present disclosure include depositing a reactive layer on top of a conductive layer by a sequential step deposition process in a single deposition chamber at room temperature. In some embodiments, the reactive layer is applied on top of the conductive layer after a predetermined period after conductive layer deposition. For example, the reactive layer may be applied on top of the conductive layer, 1 second or more after conductive layer deposition, such as 2 seconds or more, such as 5 seconds or more, such as 10 seconds or more, such as 60 seconds or more, including 100 seconds or more after conductive layer deposition. In certain instances, the substrate with applied conductive layer may be stored for a period of time before applying the reactive layer on top of the conductive layer. In certain instances, the substrate may be stored for 1 to 1000 days or longer, such as 1 to 100 days or longer, including 1 to 10 days or longer. Any storage method may be employed so long as it is sufficient to store the substrate with applied conductive layer without changing any of the desired properties of the conductive layer.

In other embodiments, the reactive layer may be applied on top of the conductive layer immediately after applying the conductive layer to the substrate. By “immediately” is meant that the reactive layer begins deposition as soon as deposition of the conductive layer has terminated. In some instances, the reactive layer may even commence for a short predetermined period of time prior to termination of conductive layer deposition resulting in a thin layer of a multicomponent layer (composed of both conductive layer material and reactive layer material) formed between the conductive layer and reactive layers.

In certain embodiments, the deposited reactive layer may be a mixture of the conductive layer material and the reactive layer material to produce a multicomponent reactive layer. For example, where the conductive layer material is silver (Ag) and the reactive layer material is silver chloride (AgCl), methods include depositing a mixture of Ag and AgCl to produce a multicomponent reactive layer. As such, the molar ratio of the Ag to AgCl in the deposited reactive layer may vary, and in some instances may range between 1:1 and 1:2.5; 1:2.5 and 1:5; 1:5 and 1:10; 1:10 and 1:25; 1:25 and 1:50; 1:50 and 1:100; 1:100 and 1:150; 1:150 and 1:200; 1:200 and 1:250; 1:250 and 1:500; 1:500 and 1:1000, or a range thereof. In other instances, the molar ratio of AgCl to Ag in the deposited reactive layer may vary, and in some instances may range between 1:1 and 1:2.5; 1:2.5 and 1:5; 1:5 and 1:10; 1:10 and 1:25; 1:25 and 1:50; 1:50 and 1:100; 1:100 and 1:150; 1:150 and 1:200; 1:200 and 1:250; 1:250 and 1:500; 1:500 and 1:1000, or a range thereof.

Accordingly, methods of the present disclosure also include simultaneously depositing conductive layer material by the high-voltage electron beam thermal evaporation and depositing reactive layer material by the low-voltage resistive heating thermal evaporation at room temperature. The rate of deposition by each component can vary depending on the molar ratio of the deposited conductive and reactive layer materials desired. For example, the rate of deposition by high-voltage electron beam thermal evaporation may range from 1 to 10 Å/s, such as 2 to 10 Å/s, such as 2 to 5 Å/s, including 2.5 Å/s and the rate of deposition by low-voltage resistive heating thermal evaporation operating concurrently may range from 1 to 15 Å/s, such as 2 to 10 Å/s, such as 2 to 5 Å/s, including 5 Å/s. As such, in some instances the ratio of the deposition rates of conductive layer material by high-voltage electron beam thermal evaporation and reactive layer material by low-voltage resistive heating thermal evaporation may vary, ranging between 1:1 and 1:2.5; 1:2.5 and 1:5; 1:5 and 1:10; 1:10 and 1:25; 1:25 and 1:50; 1:50 and 1:100; 1:100 and 1:150; 1:150 and 1:200; 1:200 and 1:250; 1:250 and 1:500; 1:500 and 1:1000, or a range thereof. In other instances, the ratio of the deposition rates of reactive layer material by low-voltage resistive heating thermal evaporation and conductive layer material by high-voltage electron beam thermal evaporation may vary, ranging between 1:1 and 1:2.5; 1:2.5 and 1:5; 1:5 and 1:10; 1:10 and 1:25; 1:25 and 1:50; 1:50 and 1:100; 1:100 and 1:150; 1:150 and 1:200; 1:200 and 1:250; 1:250 and 1:500; 1:500 and 1:1000, or a range thereof. Therefore, electrodes produced by methods as described herein may also include a conductive layer and a multicomponent reactive layer deposited on top of the conductive layer which includes reactive layer material and conductive layer material.

Aspects of the present disclosure also include monitoring the deposition of the conductive layer and reactive layer. By “monitoring” is meant that one or more properties of the conductive and reactive layers are determined and assessed in conjunction with or after deposition. Furthermore, monitoring may also include assessing/maintaining operating parameters of the deposition process such as for example, operating voltage, electron beam power, resistive heating, entraining gaseous flow, substrate configuration and angle, substrate movement, substrate surface temperature, deposition chamber pressure and temperature.

In some embodiments, methods include determining the chemical makeup of conductive and reactive layers. Determining the chemical makeup refers to the analysis of one or more of the chemical properties or components present in the conductive and reactive layers. Determining the makeup of the conductive and reactive layers may include, but is not limited to determining the chemical makeup (e.g., metal composition, amount of impurity), electrochemical properties, spectroscopic properties, and conductivity of the conductive and reactive layers. Any convenient protocol can be employed to determine the makeup of the conductive and reactive layers. Methods for analyzing the makeup of the conductive and reactive layers include, but are not limited to IR spectroscopy, UV-vis spectrophotometry, visible microscopy, electron microscopy as well as conductivity experiments.

In other embodiments, methods include determining the physical makeup of the conductive and reactive layers. Determining the physical makeup refers to the analysis of one or more physical parameters of the conductive and reactive layers. For example, the amount of material deposited, thickness, smoothness and uniformity of each layer may be assessed. Any convenient protocol can be employed to determine the physical makeup of the conductive and reactive layers. Methods for analyzing the physical makeup of the conductive and reactive layers include, but are not limited to quartz crystal microbalance, visible microscopy, electron microscopy, surface reflection analysis, contact angle studies, among others.

The conductive and reactive layers may be monitored at any phase during methods of the invention. For example, the makeup of the conductive and reactive layer may be determined immediately after deposition, respectively. In other embodiments, the makeup of the conductive and reactive layers is determined throughout the deposition process. For instances, data (i.e., thickness, conductivity, impurity content, etc.) about the conductive and reactive layers may be monitored throughout the deposition process, such by real-time data collection. In other embodiments, the conductive and reactive layers are monitored during the deposition process by collecting data at regular intervals, e.g., collecting data every 1 minute, every 5 minutes, every 10 minutes, every 30 minutes, every 60 minutes, every 100 minutes, every 200 minutes, every 500 minutes, or some other interval.

Methods of the present disclosure also include assessing the collected data. By “assessing” the collected data is meant that a human (either alone or with the assistance of a computer, if using a computer automated process initially set up under human direction), evaluates the collected data about the conductive and reactive layers and determines whether the each layer is suitable or unsuitable. For example, if after assessing that the conductive layer is suitable for applying the reactive layer, the reactive layer may be deposited on top of the conductive layer without any further adjustments. In other words, methods of these embodiments include a step of assessing the collected data to identify any desired adjustments to the conductive layer. The desired adjustments may vary in terms of goal, where in some instances the desired adjustments that ultimately result in enhanced efficiency of some desirable parameter, e.g., conductivity, smoothness, uniformity, thickness, less electrode fouling. In some instances, where each respective layer has been determined to be at least less than optimal, that layer may be further processed before proceeding to the next deposition step (e.g., applying the reactive layer on top of the conductive layer). In other instances, where the respective layer has been determined to be at least less than optimal, that layer may be further processed concurrently while proceeding to the next deposition step. If necessary, the each layer may be processed at more than one time during methods of the present disclosure.

In certain embodiments, processing may include adjusting the thickness of the deposited layer. For instance, processing the conductive or reactive layer may include increasing the thickness of the deposited layer, such as by 0.1 nm or more, such as 0.5 nm or more, such as 1.0 nm or more, such as 1.5 nm or more, such as 2.0 nm or more, such as 5 nm or more, including 10 nm or more. The thickness of part or all of each layer maybe adjusted. For example, in some embodiments, methods include increasing the thickness of the entire deposited layer (either conductive or reactive). In other embodiments, less than that entire deposited layer may be increased in thickness, such as 95% or less of the deposited layer is increased in thickness, such as 75% or less, such as 50% or less, such as 25% or less, such as 10% or less, and including 5% or less of the conductive layer is increased in overall thickness. In certain instances, specific regions on the deposited layer may be adjusted, resulting in discrete portions of the layer having varying thickness.

In other embodiments, processing may include adjusting the smoothness of the deposited layer. For instance, processing the conductive or reactive layer may include improving the smoothness of the deposited layer. As such, all or a portion of the deposited layer may be processed to improve the smoothness of the deposited layer. In some instances, discrete positions on the deposited layer may be targeted for improving smoothness.

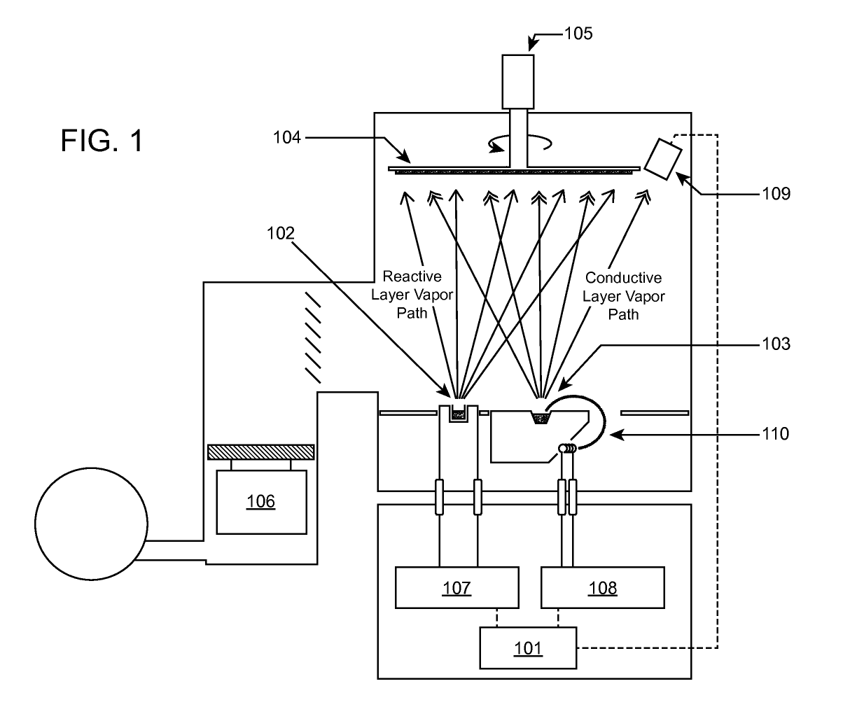

FIG. 1 depicts an example system for practicing methods of the present disclosure. The deposition chamber includes a deposition controller (101) for inputting operation and deposition parameters for both the low-voltage resistive heater and the high-voltage electron beam source. The deposition controller also includes software for controlling the sample chamber temperature and pressure as well as for communication with the deposition monitor (109) (e.g., quartz crystal microbalance) and substrate movement motor. The deposition chamber includes a low-voltage heater (e.g., resistive crucible heater) (102) and high-voltage electron beam emitter (e.g., thermionic electron beam gun) (110) and crucibles containing reactive layer material and conductive layer material (103). The deposition chamber also includes a substrate holder (104) for immobilizing the substrate during deposition and a substrate movement motor (105) configured to move the substrate during deposition, as desired. The sample chamber is also in gaseous communication to an evacuation source (106), such as a high vacuum pump (e.g., a turbomolecular pump backed by a mechanical roughing pump) for producing a reduced pressure atmosphere within the sample chamber. The sample chamber may also include a source of entrainment gases for evaporant deposition.

The low-voltage heater includes a low-voltage power supply (107), a resistive heating element and a crucible (e.g., quartz-crystal crucible) for holding the reactive layer material. The electron beam thermal evaporation component includes a high-voltage power supply (108), crucible for holding the conductive layer material and an electron beam source (e.g., a thermionic electron beam high voltage filament emitter).

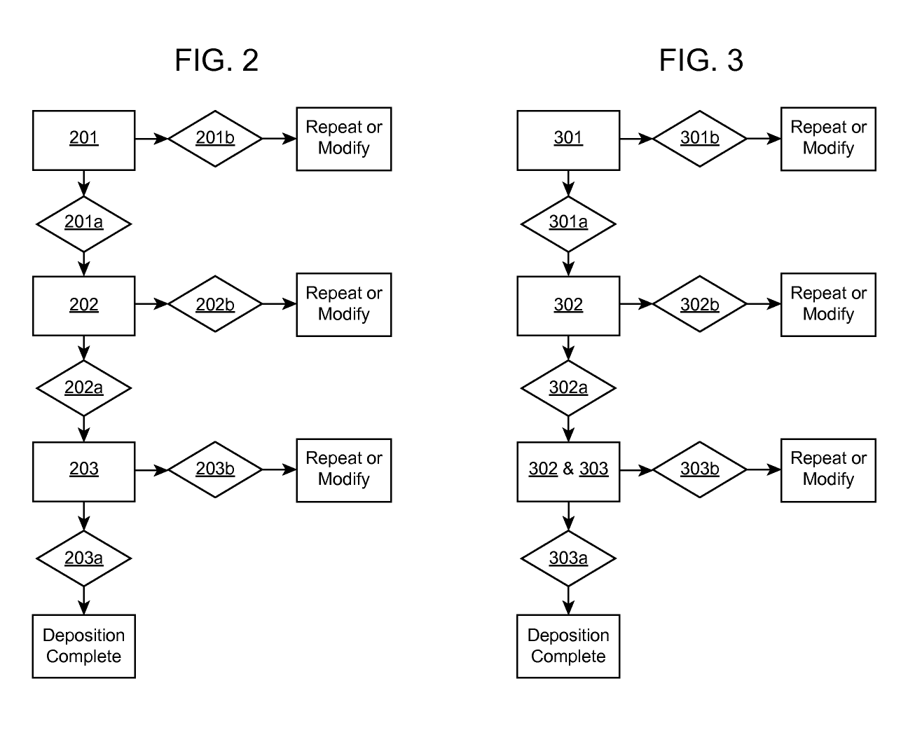

FIG. 2 illustrates a flowchart for practicing the subject methods according to certain embodiments. In the embodiment illustrated in FIG. 2, a substrate is prepared for deposition at 201. As discussed above, the substrate may be conditioned for deposition of the conductive layer, such as by corona discharge process. Surface treatment prepares the substrate surface to be more receptive (i.e., improve the deposition of the conductive layer by modifying the adhesion or surface smoothness) to the deposited conductive layer. In certain embodiments, the surface hydrophilicity is modified. In other embodiments, the surface hydrophobicity is modified. In yet other embodiments, the surface metal adhesion is modified. Preparation step 201 may be repeated as many times as necessary so long as the substrate is conditioned and ready for conductive layer deposition. In certain instances, preparing the substrate includes depositing an adhesion layer to the surface of the substrate. In these instances, the adhesion layer may be a metal adhesion layer, such as for example a chromium adhesion layer. The adhesion layer may be deposited on the surface of the substrate by high-voltage electron beam thermal evaporation, as described above. The thickness of the adhesion layer may vary, depending on the size and desired properties of the electrode. For example, the thickness of the adhesive layer may be 0.01 Å or more, such as 0.05 Å or more, such as 0.1 Å or more, such as 0.1 Å or more, such as 0.5 Å or more, such as 1 Å or more, such as 1.5 Å or more, such as 2 Å or more, such as 5 Å or more, including 10 Å or more. As desired, deposition of an adhesion layer may be repeated as many times as necessary in order to prepare for deposition of the conductive layer. As such, the thickness of all or part of the adhesion layer may be adjusted at any time before the conductive layer is deposited onto the adhesive layer. For example, all or part of the adhesion layer be increased by 0.01 Å or more, such as by 0.05 Å or more, such as by 0.1 Å or more, such as by 0.1 Å or more, such as by 0.5 Å or more, such as by 1 Å or more, such as by 1.5 Å or more, such as by 2 Å or more, such as by 5 Å or more, including by 10 Å or more. If the substrate surface or adhesion layer is determined to be suitable for deposition of the conductive layer (201a), then the conductive layer is deposited without further adjustments onto the substrate surface or adhesion layer. However, if the substrate surface or adhesion layer are determined to be unsuitable for deposition of the conductive layer (201b), then one or more of the conditioning or adhesion layer steps may be repeated or modified in order to prepare for conductive layer deposition. In certain instances, no adhesion layer is added and the conductive layer is deposited directly onto the substrate surface.

Where the substrate or adhesion layer has been determined to be suitable for deposition (after adjustments, if necessary) of the conductive layer (201a), the conductive layer is deposited on top of the adhesion layer or directly onto the substrate at step 202. As described above, the conductive layer is deposited by high-voltage electron beam thermal evaporation at room temperature. The deposition of the conductive layer is monitored throughout the deposition process, for example, by quartz crystal microbalance and deposition parameters may be adjusted at any time. As desired, the deposition of the conductive layer can be repeated as many times as necessary. For example, if the properties of the conductive layer are determined to be suitable for deposition of the reactive layer (202a), then the reactive layer is deposited without further adjustments onto the conductive layer. However, if the conductive layer is determined to be unsuitable for deposition of the reactive layer (202b), then the conductive layer may be adjusted or deposition may be repeated. For example, the thickness or uniformity of the conductive layer may be adjusted. In other instances, the smoothness of the conductive layer may be improved by depositing another layer of the conductive material.

Where the conductive layer is determined to be suitable for deposition (after adjustments, if necessary) of the reactive layer (202a), the reactive layer is deposited directly on top of the conductive layer at step 203. As described above, the reactive layer is deposited by low-voltage resistive heating thermal evaporation operating at room temperature. The reactive layer, as illustrated at step 203 is deposited immediately after deposition of the conductive layer or after a predetermined time after deposition of the conductive layer. The deposition of the reactive layer may be monitored throughout the deposition process, for example, by quartz crystal microbalance and deposition parameters may be adjusted at any time. As desired, the deposition of the reactive layer can be repeated as many times as necessary. For example, if the properties of the reactive layer are determined to be suitable for employing in an electrochemical sensor (203a), then the deposition process is terminated without any further adjustments. However, if the conductive layer is determined to be unsuitable (202b), then the reactive layer may be adjusted or deposition may be repeated. For example, the thickness or uniformity of the reactive layer may be adjusted. In other instances, the smoothness of the reactive layer may be improved by depositing another layer of the reactive layer material.

FIG. 3 illustrates a flowchart for practicing the subject methods according to another embodiment. In the embodiment illustrated in FIG. 3, a substrate is prepared for deposition at 301, in the manner discussed above in FIG. 2. As discussed above, the substrate may be conditioned for deposition of the conductive layer, such as by corona discharge process. Surface treatment conditions or prepares the substrate surface to be more receptive (i.e., improve the deposition of the conductive layer by modifying the adhesion or surface smoothness) to the applied conductive layer. In certain embodiments, the surface hydrophilicity is modified. In other embodiments, the surface hydrophobicity is modified. In yet other embodiments, the surface metal adhesion is modified. Preparation step 301 may be repeated as many times as necessary so long as the substrate is conditioned and ready conductive layer deposition. In some instances, preparing the substrate includes depositing an adhesion layer to the surface of the substrate. In these instances, the adhesion layer may be a metal adhesion layer, such as for example a chromium adhesion layer. In certain instances, no adhesion layer is added and the conductive layer is deposited directly onto the substrate surface.

Where the substrate or adhesion layer has been determined to be suitable for deposition (after adjustments, if necessary) of the conductive layer (301a), the conductive layer is deposited on top of the adhesion layer or directly onto the substrate at step 302. As described above, the conductive layer is deposited by high-voltage electron beam thermal evaporation at room temperature. The deposition of the conductive layer is monitored throughout the deposition process and deposition parameters may be adjusted at any time. As desired, the deposition of the conductive layer can be repeated as many times as necessary. For example, if the properties of the conductive layer are determined to be suitable for deposition of the reactive layer (302a), then the reactive layer is deposited without further adjustments onto the conductive layer. However, if the conductive layer is determined to be unsuitable for deposition of the reactive layer (302b), then the conductive layer may be adjusted or deposition may be repeated.

Where the conductive layer is determined to be suitable for deposition (after adjustments, if necessary) of the reactive layer (302a), the reactive layer is deposited directly on top of the conductive layer at step 303. As shown in FIG. 3, the reactive layer is a mixture of the conductive layer material (e.g., silver, Ag) and reactive layer material (e.g., silver chloride, AgCl). When the conductive layer is determined to be suitable for deposition of the reactive layer, both high-voltage electron beam thermal evaporation for depositing conductive layer material and low-voltage resistive heating thermal evaporation for depositing reactive layer material are commenced (or the high-voltage electron beam thermal evaporation remains on and low-voltage resistive heating thermal evaporation is commenced) and a reactive layer having a mixture of conductive layer material and reactive layer material are deposited onto the applied conductive layer (302 and 303). As such, in this embodiment, the high-voltage electron beam thermal evaporation for depositing conductive layer material and low-voltage resistive heating thermal evaporation for depositing reactive layer material are operated simultaneously to produce a multicomponent reactive layer (e.g., Ag and AgCl). The deposition of the multicomponent reactive layer may be monitored throughout the deposition process and deposition parameters may be adjusted at any time. For example, the deposition of one component may be increased or decreased to modify the constitution of the multicomponent reactive layer. As described above, the molar ratio of the conductive layer material (e.g., Ag) and reactive layer material (AgCl) in the deposited multicomponent reactive layer may vary, and in some instances may range between 1:1 and 1:2.5; 1:2.5 and 1:5; 1:5 and 1:10; 1:10 and 1:25; 1:25 and 1:50; 1:50 and 1:100; 1:100 and 1:150; 1:150 and 1:200; 1:200 and 1:250; 1:250 and 1:500; 1:500 and 1:1000, or a range thereof. In other instances, the molar ratio of reactive layer material (e.g., AgCl) to conductive layer material (e.g., Ag) in the deposited multicomponent reactive layer may vary, and in some instances may range between 1:1 and 1:2.5; 1:2.5 and 1:5; 1:5 and 1:10; 1:10 and 1:25; 1:25 and 1:50; 1:50 and 1:100; 1:100 and 1:150; 1:150 and 1:200; 1:200 and 1:250; 1:250 and 1:500; 1:500 and 1:1000, or a range thereof. Either the high-voltage electron beam thermal evaporation or the low-voltage resistive heating thermal evaporation may be turned off at any time during the deposition process, as desired.

### Analyte Test Strips

The present methods can be used to make a variety analyte test strips of any kind, size, or shape known to those skilled in the art; for example, FREESTYLE® and FREESTYLE LITE™ test strips, as well as PRECISION™ test strips sold by ABBOTT DIABETES CARE Inc. In addition to the embodiments specifically disclosed herein, the present methods can be employed with a wide variety of analyte test strips, e.g., those disclosed in U.S. patent application Ser. No. 11/461,725, filed Aug. 1, 2006; U.S. Patent Application Publication No. 2007/0095661; U.S. Patent Application Publication No. 2006/0091006; U.S. Patent Application Publication No. 2006/0025662; U.S. Patent Application Publication No. 2008/0267823; U.S. Patent Application Publication No. 2007/0108048; U.S. Patent Application Publication No. 2008/0102441; U.S. Patent Application Publication No. 2008/0066305; U.S. Patent Application Publication No. 2007/0199818; U.S. Patent Application Publication No. 2008/0148873; U.S. Patent Application Publication No. 2007/0068807; U.S. patent application Ser. No. 12/102,374, filed Apr. 14, 2008, and U.S. Patent Application Publication No. 2009/0095625; U.S. Pat. No. 6,616,819; U.S. Pat. No. 6,143,164; U.S. Pat. No. 6,592,745; U.S. Pat. No. 6,071,391 and U.S. Pat. No. 6,893,545; the disclosures of each of which are incorporated by reference herein in their entirety.

The present description should not be considered limited to the particular examples described above, but rather should be understood to cover all aspects as fairly set out in the attached claims. Various modifications, equivalent processes, as well as numerous structures to which the transition metal complexes may be applicable will be readily apparent to those of skill in the art upon review of the instant specification.

1-72. (canceled)

73. A method of making an electrochemical sensor comprising an electrode, the electrode comprising a conductive layer and a reactive layer, wherein the method comprises:
applying a first material on a substrate by high-voltage electron beam thermal evaporation to produce a conductive layer; and
applying a second material onto the conductive layer by low-voltage resistive thermal evaporation to produce a reactive layer,
wherein the conductive layer and the reactive layer are applied in the single chamber.

74. The method according to claim 73, wherein the first material comprises a conductive compound selected from the group consisting of silver, indium tin oxide, gold, platinum, copper, nickel, rhodium, ruthenium, ruthenium dioxide, cobalt, zinc, titanium, palladium, carbon and platinum-carbon.

75. The method according to claim 74, wherein the first material comprises silver.

76. The method according to claim 73, wherein the second material comprises silver chloride.

77. The method according to claim 73, wherein the method further comprises applying an adhesion layer to the substrate before applying the first material.

78. The method according to claim 77, wherein the adhesion layer comprises chromium.

79. The method according to claim 77, wherein the adhesion layer is applied to the substrate by high-voltage electron beam thermal evaporation.

80. The method according to claim 73, wherein the first material and the second material are applied at room temperature.

81. The method according to claim 73, wherein the second material is applied onto the surface of the conductive layer in the absence of a curing step after applying the first material.

82. The method according to claim 73, wherein the reactive layer is produced in the absence of a curing step after applying the second material.

83. The method according to claim 73, wherein the method further comprises monitoring the conductive layer and the reactive layer.

84. The method according to claim 73, wherein the reactive layer is applied on top of the conductive layer after the conductive layer has reached a predetermined thickness.

85. The method according to claim 73, wherein the conductive layer is applied at a rate of 2 Å per second to 10 Å per second.

86. The method according to claim 73, wherein the reactive layer is applied at a rate of 2 Å per second to 5 Å per second.

87. The method according to claim 73, wherein the reactive layer is applied by low-voltage resistive thermal evaporation operating at a voltage of 10V or less.

88. The method according to claim 73, wherein the method comprises rotating the substrate while applying the first material and the second material.

89. The method according to claim 73, wherein the method comprises moving the substrate laterally back-and-forth while applying the first material and the second material.

90. The method according to claim 73, wherein the applied reactive layer is maintained at room temperature.

91. The method according to claim 73, wherein the single chamber is maintained at a pressure of 5×10⁻⁶ torr or less.

92. The method according to claim 91, wherein the single chamber is maintained at a pressure of 1×10⁻⁶ torr or less.

93. The method according to claim 73, wherein the substrate is an inert substrate.

94. The method according to claim 73, wherein the method further comprises treating the substrate with a corona discharge process before to applying the first material.

95. The method according to claim 73, wherein the purity of the applied conductive layer is 99% or greater by weight.

96. The method according to claim 73, wherein the purity of the applied reactive layer is 99% or greater by weight.
